SEQUENCE LISTING (9 sequences)

SEQ ID NO: 1 - DNA, 1908 bp, synthetic construct
  source 1..1908: mol_type = other DNA
atggccaagaagtccgccgagaacggcatctactccgtgagcggggatgagaagaaggggcccctgatcgcccccggccccgatggcgcccccgccaagggggacggccccgtggggctcgggacacccggagggagactggccgtgccccctagggagacatggacacggcagatggattttatcatgagctgcgtgggcttcgccgtggggctggggaacgtgtggaggtttccctacctgtgctacaagaacggcggcggcgtgttcctgatcccctacgtgctgatcgccctggtgggcggcatccccatcttcttcctggaaatcagcctggggcagtttatgaaggctgggagtatcaacgtgtggaacatctgcccattgttcaagggcctgggctacgccagcatggtcattgtgttctactgcaacacctactacatcatggtgctggcctgggggttctactacctggtcaagagcttcacaacaaccctgccatgggccacatgcggccacacatggaacacacctgactgcgtcgagatcttccggcacgaggattgcgccaacgccagcctggccaacctgacctgcgatcagctggccgacaggaggagccccgtgatcgagttctgggagaacaaggtgctgcggctgtccggcgggctggaggtgcccggcgccctgaactgggaggtgaccctctgcctgctggcctgctgggtgctggtgtacttttgcgtgtggaagggggtgaagtccaccggcaagatcgtgtacttcaccgccacattcccttacgtggtgctggtggtcctgctggtcaggggcgtgctcctgcccggagccctggacggcatcatctactacctgaagcccgattggagcaagctggggtccccccaggtgtggatcgatgccgggacacagatcttcttttcctacgccattggactcggcgccctgacagccctgggaagttacaacaggtttaacaacaactgctacaaggatgccatcattttggctctgattaacagcggaactagcttctttgcagggttcgtcgtgtttagcattctgggcttcatggccgctgaacagggagtgcacatttccaaggtggctgaatccggccccggcctggccttcatcgcctaccccagggccgtgacactgatgcccgtggcccccctgtgggccgccctgttcttcttcatgctgctgctgctgggcctggacagccagttcgtgggggtggaggggttcatcacaggcctgctggatctgctgccagccagctactacttccggttccagcgggagatcagcgtggccctgtgctgcgccctgtgcttcgtgatcgatctgagcatggtgaccgacggggggatgtacgtgttccagctgttcgactactacagcgcctccgggacaaccctgctgtggcaggccttctgggagtgcgtggtggtggcctgggtgtacggggccgacaggttcatggatgatattgcctgcatgatcggctaccggccctgcccctggatgaagtggtgctggagcttcttcacacccctggtgtgcatgggcatcttcatcttcaacgtggtgtactacgagcccctggtgtacaacaacacctacgtgtacccatggtggggggaggccatgggctgggccttcgccctgagcagcatgctgtgcgtgcccctgcacctgctggggtgcctgctgagggccaaggggacaatggccgagcggtggcagcacctgacacagcccatctgggggctgcaccacctggagtacagggcccaggatgccgacgtgaggggcctgacaaccctgaccccagtgagtgaatctagtaaggtagtagtagttgagtcagtgatgtga

SEQ ID NO: 2 - DNA, 2432 bp, synthetic construct
  source 1..2432: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgagcagaattcgccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggccaagaagtccgccgagaacggcatctactccgtgagcggggatgagaagaaggggcccctgatcgcccccggccccgatggcgcccccgccaagggggacggccccgtggggctcgggacacccggagggagactggccgtgccccctagggagacatggacacggcagatggattttatcatgagctgcgtgggcttcgccgtggggctggggaacgtgtggaggtttccctacctgtgctacaagaacggcggcggcgtgttcctgatcccctacgtgctgatcgccctggtgggcggcatccccatcttcttcctggaaatcagcctggggcagtttatgaaggctgggagtatcaacgtgtggaacatctgcccattgttcaagggcctgggctacgccagcatggtcattgtgttctactgcaacacctactacatcatggtgctggcctgggggttctactacctggtcaagagcttcacaacaaccctgccatgggccacatgcggccacacatggaacacacctgactgcgtcgagatcttccggcacgaggattgcgccaacgccagcctggccaacctgacctgcgatcagctggccgacaggaggagccccgtgatcgagttctgggagaacaaggtgctgcggctgtccggcgggctggaggtgcccggcgccctgaactgggaggtgaccctctgcctgctggcctgctgggtgctggtgtacttttgcgtgtggaagggggtgaagtccaccggcaagatcgtgtacttcaccgccacattcccttacgtggtgctggtggtcctgctggtcaggggcgtgctcctgcccggagccctggacggcatcatctactacctgaagcccgattggagcaagctggggtccccccaggtgtggatcgatgccgggacacagatcttcttttcctacgccattggactcggcgccctgacagccctgggaagttacaacaggtttaacaacaactgctacaaggatgccatcattttggctctgattaacagcggaactagcttctttgcagggttcgtcgtgtttagcattctgggcttcatggccgctgaacagggagtgcacatttccaaggtggctgaatccggccccggcctggccttcatcgcctaccccagggccgtgacactgatgcccgtggcccccctgtgggccgccctgttcttcttcatgctgctgctgctgggcctggacagccagttcgtgggggtggaggggttcatcacaggcctgctggatctgctgccagccagctactacttccggttccagcgggagatcagcgtggccctgtgctgcgccctgtgcttcgtgatcgatctgagcatggtgaccgacggggggatgtacgtgttccagctgttcgactactacagcgcctccgggacaaccctgctgtggcaggccttctgggagtgcgtggtggtggcctgggtgtacggggccgacaggttcatggatgatattgcctgcatgatcggctaccggccctgcccctggatgaagtggtgctggagcttcttcacacccctggtgtgcatgggcatcttcatcttcaacgtggtgtactacgagcccctggtgtacaacaacacctacgtgtacccatggtggggggaggccatgggctgggccttcgccctgagcagcatgctgtgcgtgcccctgcacctgctggggtgcctgctgagggccaaggggacaatggccgagcggtggcagcacctgacacagcccatctgggggctgcaccacctggagtacagggcccaggatgccgacgtgaggggcctgacaaccctgaccccagtgagtgaatctagtaaggtagtagtagttgagtcagtgatgtgagcagtcgacgccaataaagagctcagatgcatcgatcagagtgtgttggttttttgtgtgccactccctctctgcgcgctcgctcgctcactgaggccgggcgaccaaaggtcgcccgacgcccgggctttgcccgggcggcctcagtgagcgagcgagcgcgcagagaggga

SEQ ID NO: 3 - DNA, 173 bp, synthetic construct
  source 1..173: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgagcagaattcgcc

SEQ ID NO: 4 - DNA, 178 bp, synthetic construct
  source 1..178: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccacc

SEQ ID NO: 5 - DNA, 173 bp, synthetic construct
  source 1..173: mol_type = other DNA
gcagtcgacgccaataaagagctcagatgcatcgatcagagtgtgttggttttttgtgtgccactccctctctgcgcgctcgctcgctcactgaggccgggcgaccaaaggtcgcccgacgcccgggctttgcccgggcggcctcagtgagcgagcgagcgcgcagagaggga

SEQ ID NO: 6 - DNA, 23 bp, synthetic construct
  source 1..23: mol_type = other DNA
ggacccaaggactacctcaaggg

SEQ ID NO: 7 - DNA, 22 bp, synthetic construct
  source 1..22: mol_type = other DNA
agggcacctccatctcggaaac

SEQ ID NO: 8 - DNA, 20 bp, synthetic construct
  source 1..20: mol_type = other DNA
cagcctggggcagtttatga

SEQ ID NO: 9 - DNA, 20 bp, synthetic construct
  source 1..20: mol_type = other DNA
acaatgaccatgctggcgta

(19) United States
(12) Patent Application Publication
(10) Pub. No.: US 2026/0356899 A1
(43) Pub. Date: Nov. 12, 2026

(54) SOLUTE CARRIER FAMILY 6 MEMBER 8 VECTORS AND METHODS FOR USE THEREOF

(71) Applicants: GENCORREX THERAPIES INC., Kingston (CA); THE BOARD OF REGENTS OF THE UNIVERSITY OF TEXAS SYSTEM, Austin, TX (US)

(72) Inventors: Jagdeep Walia, Kingston (CA); Steven Gray, Austin, TX (US)

(21) Appl. No.: 19/484,122

(22) PCT Filed: May 17, 2024
(86) PCT No.: PCT/CA2024/050664

§ 371 Date: Nov. 13, 2025

Related U.S. Application Data
(60) Provisional application No. 63/467,405, filed on May 18, 2023.

(51) Int. Cl. C07K 14/47; A61K 38/17; A61K 48/00; C12N 15/86

(52) U.S. Cl. CPC C07K 14/47; A61K 38/1709; A61K 48/0033; A61K 48/005; C12N 15/86; C12N 2750/14143

(57) ABSTRACT

This disclosure relates to nucleic acid constructs for expression of Solute Carrier Family 6 Members (SLC6A8) protein, and viral vectors comprising said constructs useful for the treatment of cerebral creatine transporter deficiency (CCDS1). Also provided are methods and uses of the vectors disclosed herein for the treatment of CCDS1.

### RELATED APPLICATION

This application claims benefit of U.S. Provisional Patent Application Ser. No. 63/467,405 filed May 18, 2023, incorporated herein by reference in its entirety.

### INCORPORATION OF SEQUENCE LISTING

A computer readable form of the Sequence Listing “29669-P72032PC00_SequenceListing.xml” (13,720 bytes), created on May 16, 2024, is herein incorporated by reference.

### FIELD

The present disclosure relates to the development of a nucleic acid construct comprising a transgene encoding the DNA sequence for a Solute Carrier Family 6 Member 8 (SLC6A8) protein operably linked to a promoter, and a transcription termination site; vectors comprising said nucleic acid constructs; pharmaceutical compositions comprising said vector; and vectors or compositions for use in the treatment of cerebral creatine transporter deficiency (CCDS1).

### INTRODUCTION

Creatine is a small molecule phosphate donor that increases the energy storage capability of the cell. Creatine is predominantly known as a popular athletic supplement known to improve the duration users can sustain high intensity exercise. More recently creatine has been shown to play a role in a vast array of processes including immune differentiation, cancer proliferation, embryonic development and perhaps most notably as a necessity for healthy brain function.

The synthesis of creatine begins with the conversion of arginine and glycine into guanidinoacetate (GAA) via the enzyme Arginine:Glycine Amidinotransferase (AGAT)¹ (FIG. 1). GAA is then converted to creatine after methylation by Guanidinoacetate Methyltransferase (GAMT) in the presence of methyl donor S-adenosylmethionine (SAM)². GAA is a toxic intermediate and thought to play a large role in the pathology of patients with GAMT deficiency³. Solute Carrier Family 6 Member 8 (SLC6A8, AKA creatine transporter or CTR) is an Na⁺Cl⁻ dependent transporter of creatine and to a lesser degree GAA and is responsible cellular intake of creatine⁴⁻⁶. Creatine is then converted into creatinine via a spontaneous process before being released through urine. SLC6A10 is a second creatine transporter originally thought to be solely expressed in the testis, however, it is also expressed at least to a limited degree in the brain^{7,8}.

Creatine phosphorylation and dephosphorylation are controlled through the creatine kinase system. In short, during periods of energy surplus creatine kinase phosphorylates creatine by transferring a phosphate group from ATP storing the energy as phosphocreatine. During periods of energy stress creatine kinase can regenerate ATP through the phosphorylation of ADP using phosphocreatine as the phosphate donor. There are a large variety of creatine kinase isoforms and which can be both cell/tissue specific as well as separated into mitochondrial and cytosolic isoforms⁹.

SLC6A8 is also present at the blood brain barrier (BBB) and allows some limited uptake of creatine from the periphery, however, the transport of creatine across the BBB appears to be minimal¹⁰. Creatine is also obtained through diet though the relative amount is dependent upon animal product consumption and supplementation”.

### Primary Creatine Deficiency

Cerebral creatine transporter deficiency (OMIM #300352, CCDS1, SLC6A8 deficiency) is an X linked genetic disorder induced by loss of function mutations to SLC6A8 which result in the inability for cells to uptake creatine from the periphery leading to severely depleted or absence of creatine in the central nervous system (CNS)^{5,12}. Elevated guanidinoacetate is possibly due to the different localization of cells that express AGAT and GAMT and in the absence of the transporter guanidinoacetate can accumulate¹³. Alternatively reduced creatine concentration may induce AGAT expression production leading to increased GAA accumulation. Peripheral creatine concentrations and phenotypes vary wildly but tend to show reduced creatine in specific cell types such as muscle cells and an organism wide increased metabolism^{14,15}. Disease manifestation occurs during early childhood (OMIM #300352-SLC6A8) and consists of a wide range of symptoms with a large variation in presentation including but not limited to seizures, intellectual disability, developmental delays, and motor function impairment^{5,15}. SLC6A8 deficiency is emerging as a relatively common genetic disease, likely in part due to it being X linked and thought to cause between 1-2% of all X-linked intellectual disability^{16,17}. Due to its recent discovery and wide range of symptom presentation it may also be underdiagnosed in the population. There are currently no treatment options available in the clinic for SLC6A8 deficiency as creatine supplementation is ineffective without a transporter.

Murine models of SLC6A8 deficiency show a very similar biochemical phenotype to humans with a severely decreased creatine concentration and increased guanidinoacetate concentration in the CNS, and elevated creatine to creatinine ratio in plasma^{18,19}. In comparison, to other murine models of creatine deficiency SLC6A8 knock out (KO) mouse models display much more severe behavioral abnormalities such as decreased performance in novel object recognition tasks and Morris water maze²⁰. Spontaneous alteration in the Y maze, a measure of working memory has been shown to be affected in multiple different KO mouse models and this phenotype is even conserved in rat models of the disease^{18,19,21-23}.

### Adeno Associated Viral Vectors

Adeno associated virus (AAV) is an effective vector for gene replacement therapies due to their efficient delivery and safety profile and has now been approved for the treatment of multiple diseases²⁴. AAV serotype 9 (AAV9) has shown to have the most effective neuronal transduction of the wild type (WT) AAV capsids as well as the capability to cross the blood brain barrier²⁵. AAVs can be either self complimentary (scAAV) or single stranded (ssAAV). scAAV have been shown to have improved gene expression as compared to ssAAV as they do not require second strand synthesis which can be a rate limiting step during gene expression^{26,27}. The draw back to the use of scAAV is the packaging capacity is roughly half that of ssAAV vectors²⁸.

### SUMMARY

The present disclosure relates to nucleic acid constructs encoding functional SLC6A8 protein operably linked to a promoter and transcription termination site, as well as viral vectors comprising said nucleic acid constructs for therapeutic replacement of dysfunctional SLC6A8 protein. This disclosure also relates to the production of AAV vectors including nucleic acids encoding the SLC6A8 protein.

The present inventors have tested the first CNS-directed, AAV9-based gene therapy for the treatment of CCDS1. It was found that delivery of SLC6A8 plasmid DNA to cellular models of SLC6A8-deficiency effectively restored creatine levels. In murine models of CCDS1, treatment with scAAV9.JeT.SLC6A8 delivered intracerebroventricularly, resulted in increased creatine levels throughout the mid-section of the brain. Overall, it was found that scAAV9.JeT.SLC6A8 represents a promising gene therapy approach to treating CCDS1.

Accordingly, the present disclosure provides a nucleic acid construct comprising a promoter, a transcription termination site, and a nucleotide sequence encoding a SLC6A8 protein. It further provides a viral vector comprising said nucleic acid construct and methods of treating and preventing CCDS1 in a subject.

One aspect of the disclosure includes a nucleic acid construct comprising a nucleotide sequence encoding a SLC6A8 protein operably linked to a promoter and a transcription termination site.

In an embodiment, the nucleotide sequence encoding a SLC6A8 protein has a sequence as set forth in SEQ ID NO:1 or functional variants thereof.

In an embodiment, the nucleic acid sequence is at least 50%, at least 60%, at least 70%, at least 80%, at least 90%, at least 95%, or at least 99% identical to the nucleic acid sequence of SEQ ID NO: 1, and wherein the protein encoded by said nucleic acid sequence retains SLC6A8 activity.

In an embodiment, the SLC6A8 protein has an amino acid sequence that is at least 50%, at least 60%, at least 70%, at least 80%, at least 90%, at least 95%, or at least 99% identical to the protein encoded by SEQ ID NO: 1, and which retains SLC6A8 activity.

In an embodiment, the promoter is a constitutive promoter or a tissue- or cell-specific promoter.

In an embodiment, the promoter is selected from the group consisting of: chicken beta actin (CBA), chicken β-actin hybrid (CBh), CMV early enhancer (CAG), Elongation Factor 1α (eF-1a), simian virus 40 early promoter (SV40), human phosphoglycerate kinase 1 (PGK), cytomegalovirus immediate-early promoter (CMV), human β-actin (hACTB) and JeT synthetic promoter.

In an embodiment, the nucleic acid construct comprises a sequence set forth in SEQ ID NO: 2, or a functional variant thereof.

An aspect includes a viral vector comprising a nucleic acid construct described herein.

In an embodiment, the viral vector is an AAV vector, optionally AAV1, AAV2, AAV5, AAV6, AAV7, AAV8, and AAV9, or a derivative thereof.

An aspect includes a pharmaceutical composition comprising a nucleic acid construct or viral vector described herein and a pharmaceutically acceptable carrier or diluent for example, but not limited to, liposomes and lipid or polymer nanoparticles.

An aspect includes a method of treating or preventing cerebral creatine transporter deficiency (CCDS1) in a subject in need thereof, comprising administering to the subject a therapeutically effective amount of a viral vector or pharmaceutical composition described herein.

Another aspect includes a use of the viral vector or pharmaceutical composition described herein for the treatment or prevention of CCDS1.

A further aspect includes a use of the viral vector or pharmaceutical composition described herein for the manufacture of a medicament for the treatment or prevention of CCDS1.

Another aspect includes the viral vector or pharmaceutical composition described herein for use in the treatment or prevention of CCDS1.

In an embodiment, the viral vector or pharmaceutical composition is formulated for intravenous, intrathecal or intracerebroventricular injection.

An aspect of the disclosure includes a kit comprising the nucleic acid construct, the viral vector or the pharmaceutical composition described herein and instructions for use thereof.

These and other features and advantages of the present disclosure will become apparent from the following detailed description taken together with the accompanying drawings. It should be understood, however, that the detailed description and specific examples, while indicating preferred implementations of the present disclosure, are given by way of illustration only, since various changes and modifications within the spirit and scope of the disclosure will become apparent to those of skill in the art from this detailed description.

### DRAWINGS

Further objects, features and advantages of the disclosure will become apparent from the following detailed description taken in conjunction with the accompanying figures showing illustrative embodiments of the disclosure, in which:

FIG. 1 is a schematic that shows the synthesis transport and catabolism of creatine. Creatine is synthesized by AGAT from glycine (GLY) and arginine (ARG) to form GAA and Ornithine (ORN). GAA is then methylated by GAMT using SAM as a methyl donor. Creatine can the be phosphorylated or dephosphorylated by CK depending on the energy state of the cell. Creatine is unable to cross cellular membranes and therefore requires active transport by SLC6A8 to enter cells, GAA can also be transported by SLC6A8 although with a lower affinity than creatine. Creatine degradation occurs via a spontaneous conversion to creatinine where it can then be released through urine. A large amount of creatine is obtained through dietary intake, however, GATM (gene encoding AGAT) has been shown to undergo negative feedback in the presence of creatine.

FIG. 2 demonstrates that in vitro transfection of codon optimized SLC6A8 in KO patient fibroblasts induces creatine uptake (FIG. 2A—creatine concentration; FIG. 2B-Creatinine concentration and FIG. 2C—Guanidinoacetate, GAA). Creatine analyte concentrations were determined using liquid chromatography mass spectrometry and normalized to tissue mass. (Dunnett T3 MC test* P<0.05, **p<0.01, ***P<0.001 ****p<0.0001).

FIG. 3 demonstrates that day-0 intracerebroventricular (ICV) delivery of 0.625e10 to 1.0e11 vg of scAAV9.JeT.SLC6A8. ICV injections significantly increase body mass (FIG. 3A), creatine (FIG. 3B) and creatinine (FIG. 3C) relative to vehicle controls in the midsection of the brain. FIG. 3D shows that guanidinoacetate (GAA) levels were not decreased relative to WT but still 100-fold lower than seen in GAMT deficient mice. Mice were euthanized at 5-weeks after delivery and brain creatine, arginine, creatinine and Guanidinoacetate (GAA) were determined using liquid chromatography mass spectrometry and normalized to tissue mass. Both creatine and creatinine were significantly elevated in treated KO mice relative to KO counterparts (Dunnett T3 MC test* P<0.05, **p<0.01, ***P<0.001 ****p<0.0001).

FIG. 4 demonstrates that postnatal day-0 intracerebroventricular delivery of 2.5e10 vg of scAAV9.JeT.SLC6A8 significantly increases body mass (FIG. 4A) and decreased hyperactivity (FIG. 4B). Were placed into DVC caging 22 week after injections and dark phase movement was recorded for 14 days. At 24 weeks of age mice were weighed directly before euthanization. (Dunnett T3 MC test* P<0.05, **p<0.01, ***P<0.001 ****p<0.0001).

FIG. 5 demonstrates that postnatal day-0 intracerebroventricular delivery of 2.5e10 vg of scAAV9.JeT.SLC6A8 significantly increases creatine (FIG. 5A) and creatinine (FIG. 5C) for at least 24 weeks in the midsection of the brain. Arginine (FIG. 5B) and GAA (FIG. 5D) had no significant differences between cohorts. Mice were given immunosuppression of rapamycin and prednisone beginning at 5 weeks of age for 16 weeks. Creatine analyte concentrations were determined using liquid chromatography mass spectrometry and normalized to tissue mass. (Dunnett T3 MC test* P<0.05, **p<0.01, ***P<0.001 ****p<0.0001).

FIG. 6 demonstrates that postnatal day-0 intracerebroventricular delivery of 2.5e10 vg of scAAV9.JeT.SLC6A8 significantly increases creatine (FIG. 6A) and creatinine (FIG. 6C) for at least 24 weeks in the caudal section of the brain. Arginine (FIG. 6B) had no significant differences between cohorts however, and GAA (FIG. 6D) was elevated in both KO cohorts. Mice were given immunosuppression of rapamycin and prednisone beginning at 5 weeks of age for 16 weeks. Creatine analyte concentrations were determined using liquid chromatography mass spectrometry and normalized to tissue mass. (Dunnett T3 MC test* P<0.05, **p<0.01, ***P<0.001 ****p<0.0001).

FIG. 7 demonstrates biodistribution of P0 ICV 2.5e10 vg of scAAV9.JeT.SLC6A8 24 post injection in KO mice. Mice were given immunosuppression of rapamycin and prednisone beginning at 5 weeks of age for 16 weeks. (Dunnett T3 MC test* P<0.05, **p<0.01, ***P<0.001 ****p<0.0001).

FIG. 8 demonstrates that intrathecal delivery of 7.5e 11vg of scAAV9.JeT.SLC6A8 at 6 weeks of age significantly increases creatine (FIG. 8A), creatinine (FIG. 8B) at 13 weeks of age in the midsection of the brain. GAA (FIG. 8C) appears slightly elevated in both treated and untreated mice. Mice were given immunosuppression of rapamycin and prednisone beginning at 5 weeks of age until endpoint. Creatine analyte concentrations were determined using liquid chromatography mass spectrometry and normalized to tissue mass. (Dunnett T3 MC test* P<0.05, **p<0.01. ***P<0.001 ****p<0.0001).

FIG. 9 demonstrates that intrathecal delivery of 7.5e 11vg of scAAV9.JeT.SLC6A8 at 6 weeks of age significantly increases creatine (FIG. 9A), creatinine (FIG. 9B) at 13 weeks of age in the caudal section of the brain. GAA (FIG. 9C) appears slightly elevated in both treated and untreated mice. Mice were given immunosuppression of rapamycin and prednisone beginning at 5 weeks of age until endpoint. Creatine analyte concentrations were determined using liquid chromatography mass spectrometry and normalized to tissue mass. (Dunnett T3 MC test* P<0.05, **p<0.01, ***P<0.001 ****p<0.0001).

FIG. 10 demonstrates that intrathecal delivery of 7.5e 11vg of scAAV9.JeT. SLC6A8 at 6 weeks of age significantly increases both body mass (FIG. 10A) and performance in grip strength at 13 of age (FIG. 10B). Mice were given immunosuppression of rapamycin and prednisone beginning at 5 weeks of age until endpoint. Grip strength is evaluated by the ability to carry progressively heavier weights for up to three seconds (Dunnett T3 MC test* P<0.05, **p<0.01, ***P<0.001 ****p<0.0001).

FIG. 11 demonstrates that intrathecal delivery of 7.5e 11vg of scAAV9.JeT.SLC6A8 at 6 weeks of age significantly increases creatine (FIG. 11A), creatinine (FIG. 11B) at 24 weeks of age in the midsection of the brain. GAA (FIG. 11C) appears slightly elevated in both treated and untreated mice. Mice were given immunosuppression of rapamycin and prednisone beginning at 5 weeks of age until endpoint. Creatine analyte concentrations were determined using liquid chromatography mass spectrometry and normalized to tissue mass. (Dunnett T3 MC test* P<0.05, **p<0.01, ***P<0.001 ****p<0.0001).

FIG. 12 demonstrates that intrathecal delivery of 7.5e 11vg of scAAV9.JeT.SLC6A8 at 6 weeks of age significantly increases creatine (FIG. 12A), creatinine (FIG. 12B) at 6-months post-injection in the caudal section of the brain. GAA (FIG. 12C) appears slightly elevated in both treated and untreated mice. Mice were given immunosuppression of rapamycin and prednisone beginning at 5 weeks of age until endpoint. Creatine analyte concentrations were determined using liquid chromatography mass spectrometry and normalized to tissue mass. (Dunnett T3 MC test* P<0.05, **p<0.01, ***P<0.001 ****p<0.0001).

FIG. 13 demonstrates that intrathecal delivery of 7.5e 11vg of scAAV9.JeT.SLC6A8 at 6 weeks of age significantly increases body mass (FIG. 13A) and decreases hyperactivity (FIG. 13B). Mice were placed into DVC caging 22 weeks after injections and dark phase movement was recorded for 14 days. At 24 weeks of age mice were weighed directly before euthanization. (Dunnett T3 MC test *p<0.05, **p<0.01, ***p<0.001, ****p<0.0001).

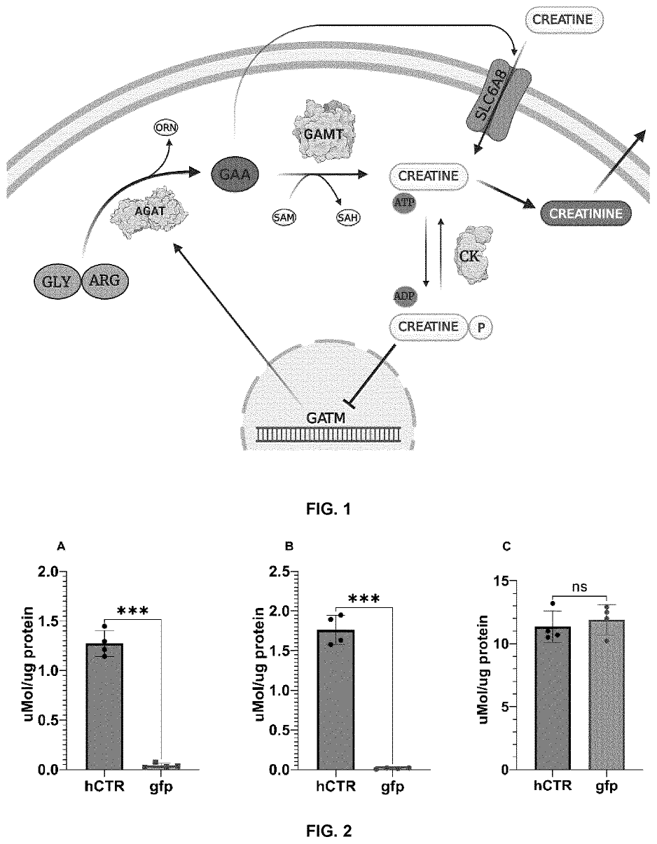

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. GENERAL DEFINITIONS

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

All numerical values herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to inclusive or be open-ended, i.e., to mean including but not limited to, and do not exclude additional, unrecited elements or process steps. Only the transitional phrases “consisting of” and “consisting essentially of” shall be closed or semi-closed transitional phrases, respectively.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular any feature described herein may be combined with any other feature or features described herein.

### II. NUCLEIC ACID CONSTRUCTS AND VIRAL VECTORS

Described herein is a nucleic acid construct comprising a nucleotide sequence encoding a SLC6A8 protein, operably linked to a promoter and a transcription termination site. As shown herein, delivery of such nucleic acid constructs via adeno-associated viral vectors results in increased creatine expression, improved body mass and decreased hyperactivity in mouse models of CCDS1. Accordingly, one aspect of the disclosure includes a nucleic acid construct comprising a nucleotide sequence encoding a SLC6A8 protein operably linked to a promoter and a transcription termination site.

The term “nucleic acid construct of the disclosure” as used herein refers to a nucleic acid molecule comprising an expression cassette, the expression cassette comprising a DNA sequence encoding a SLC6A8 protein operably linked to a promoter and a transcription termination site. In an embodiment, the DNA sequence encoding a SLC6A8 protein comprises a known SLC6A8 nucleotide sequence. In an embodiment, the DNA sequence encoding a SLC6A8 protein comprises a nucleotide sequence set forth in SEQ ID NO:1 or a functional variant thereof.

The term “Solute Carrier Family 6 Member 8” protein or “SLC6A8” also known as creatine transporter or CTR, as used herein refers to a transport protein which functions to transport creatine into and out of cells. Defects in this gene have been implicated in X-lined creatine deficiency syndrome. Transcript variants due to encoding different isoforms have been described for this gene. In an embodiment, the nucleotide and amino acid sequence of human SLC6A8 can be found at GenBank gene ID: 6535 and UniProt P48029.

The term “SLC6A8 activity” as used herein refers to a protein that is known to act as a Na⁺Cl⁻ dependent transporter for creatine into and out of the cell.

The term “nucleic acid molecule” and its derivatives, as used herein, are intended to include unmodified DNA or RNA or modified DNA or RNA. For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “operably linked” as used herein refers to a relationship between two components that allows them to function in an intended manner. For example, where a coding sequence is operably linked to a promoter, the promoter actuates expression of the coding sequence.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters for the expression cassette will be known to the skilled person. In some embodiments, the promoter is an inducible promoter. Examples of inducible promoters include, without limitation, a tetracycline response element (TRE) (e.g. Tet-ON or Tet-OFF systems), ponA-inducible expression systems (Agilent Technologies), or cumate-inducible promoters such as CuO (System Biosciences). In some embodiments, the promoter is a constitutive promoter. Examples of constitutive promoters include human Ubiquitin C (UBC), human Elongation Factor 1α (EF1A), human phosphoglycerate kinase 1 (PGK), simian virus 40 early promoter (SV40) (GeneBank accession number J02400.1), cytomegalovirus immediate-early promoter (CMV), chicken β-Actin promoter coupled with CMV early enhancer (CAG), chicken β-actin hybrid (CBh) and EF1-HTLV. In some embodiments, the promoter is a tissue- or cell-specific promoter. In an embodiment, the promoter is a synthetic promoter such as JeT.

The term “transcription termination site” as used herein refers generally to a polyadenylation signal (pA) that terminates transcription of messenger RNA (mRNA). As used herein, the phrase “polyadenylation signal” refers to sequences from various genes that can be added to mammalian vectors to ensure proper mRNA processing and stability. For example, a 100-200 nucleotide polyadenylate tail can be added to the 3′ end of a coding sequence to protect mRNA from degradatory action of phosphatases and nucleases. Suitable pAs may be derived from any organism and are known to the skilled person. Examples of pA signals include, without limitation, rabbit beta-globin pA (GeneBank accession number K03256), SV40 late polyA, and hGH polyA and strong bovine growth hormone pA (BGHpA).

The term “functional variant” as used herein includes modifications of the nucleic acid or polypeptide sequences disclosed herein that perform substantially the same function as the nucleic acid molecules or polypeptides disclosed herein in substantially the same way. For example, the functional variant may comprise sequences having at least 50%, or at least 60%, or at least 70%, or at least 80%, or at least 90%, or at least 95%, or at least 99% sequence identity to the sequences disclosed herein. In the case of nucleic acids, functional variants include nucleotide sequences that hybridize to the nucleic acid sequences set out above, under at least moderately stringent hybridization conditions, optionally stringent hybridization conditions, or the functional variant nucleic acid sequences may comprise degenerate codon substitutions or codon-optimized nucleic acid sequences. In the case of polypeptides, the functional variant may also comprise conservatively substituted amino acid sequences of the sequences disclosed herein.

In an embodiment, the functional variant sequences comprise sequences having at least 50%, or at least 60%, or at least 70%, or at least 80%, or at least 90%, or at least 95%, or at least 99% sequence identity to the sequences disclosed herein.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In one embodiment, the functional variants include nucleotide sequences that hybridize to the nucleic acid sequences described herein, under at least moderately stringent hybridization conditions, optionally stringent hybridization conditions.

With reference to nucleic acids, the terms “anneal” and “hybridize” as used herein refer to the ability of a nucleic acid to non-covalently interact with another nucleic acid through base-pairing. The terms “complementary” or “complementary nucleic acid” refer to a nucleic acid or a portion of a nucleic acid that is able to anneal with a nucleic acid of a given sequence. In some cases, this is referred to as the “reverse complement” of a given sequence.

By “at least moderately stringent hybridization conditions” it is meant that conditions are selected which promote selective hybridization between two complementary nucleic acid molecules in solution. The term “at least moderately stringent hybridization conditions” encompasses stringent hybridization conditions and moderately stringent hybridization conditions. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5 °C-16.6 (Log 10 [Na+])+0.41(% (G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1 °C decrease in Tm, for example if nucleic acid molecules are sought that have a >95% identity, the final wash temperature will be reduced by about 5 °C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm—5 °C based on the above equation, followed by a wash of 0.2×SSC/0.1% SDS at 60 °C. Moderately stringent hybridization conditions include a washing step in 3×SSC at 42 °C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

In another embodiment, the functional variant nucleic acid sequences comprise degenerate codon substitutions or codon-optimized nucleic acid sequences. The term “degenerate codon substitution” as used herein refers to variant nucleic acid sequences in which the second and/or third base of a codon is substituted with a different base that does not result in a change in the amino acid sequence encoded therein. The term “codon-optimized” as used herein refers to a variant nucleic acid molecule comprising one or more degenerate codon substitutions that reflect the codon usage bias of a particular organism. Accordingly, in an embodiment, the nucleic acid construct disclosed herein comprises a codon-optimized or degenerate nucleotide sequence of SEQ ID NO:1.

In an embodiment, the nucleic acid construct disclosed herein comprises a nucleic acid molecule that encodes a polypeptide having an amino acid sequence that is at least 50%, at least 60%, at least 70%, at least 80%, at least 90%, at least 95%, or at least 99% identical to the protein encoded by SEQ ID NO:1, and which retains SLC6A8 activity.

In an embodiment, the nucleic acid construct further comprises, an enhancer, a post-transcription regulatory sequence, one or more sequences that facilitate incorporation of the nucleic acid into a viral particle and/or integration into the host genome, or any combination thereof, operably linked to the nucleic acid encoding the SLC6A8 protein. Post transcriptional regulatory sequences include, for example, without limitation, sequences of nucleotides that when placed in an AAV transfer plasmid results in the increased or decreased expression of the transgene. As used herein, the phrase “enhancer” refers to a sequence of nucleotides that argument the activity of a promoter in an orientation, position, and distance-dependent manner. Enhancers play a significant role in the regulation of tissue-specific gene expression in high eukaryotes but have been repurposed for use in recombinant DNA technologies to impact the transcriptional activity of an associated promoter. Typically, a trans-acting gene regulatory protein binds the enhancer in order to affect transcriptional activity of the associated promoter.

In some embodiments, the nucleic acid construct comprises a sequence set forth in SEQ ID NO: 2, or a functional variant thereof.

### Viral Vectors

Also described herein is a viral construct comprising a nucleic acid construct described herein. Viral constructs are made of DNA or RNA and they contain some of the genetic material of the viruses they are derived from (such as lentivirus, retrovirus, AAV and adenoviruses). For example, viral constructs may include sequences that facilitate incorporation of the nucleic acid into a viral particle and/or integration into the host genome. In some embodiments, the viral construct may include inverted terminal repeats (ITRs) for example from an AAV such as AAV9, or other viral sequences. Viral constructs have been modified to carry and to deliver a gene of interest that will produce a protein or an RNA of interest and can be used for example for the treatment of diseases by gene therapy. Suitable viral constructs are known in the art and depend on the type of viral vectors and viruses being used.

One aspect of the disclosure is a viral vector comprising a nucleic acid construct disclosed herein. Replication incompetent viral vectors are particularly useful in gene therapy applications as they allow for efficient transduction of delivery of a transgene to target tissues. Differences between viral vectors include availability of tropisms, packaging capacity, safety, and transduction efficiencies in different tissues.

The term “viral vector” as used herein is intended to include viral particles or virus-like particles capable of transduction of a target cell. Common viral vectors include, but are not limited to, HIV-derived lentiviral vectors, retroviral vectors, adenoviral vectors, and recombinant adeno-associated virus (AAV) vectors. Other viral vectors may be derived from rhabdovirus (such as vesicular stomatitis virus (VSV)), or herpes virus (such CMV and HSV-1). Typical components of the viral vector are the structural components of the viral particle, such as the proteins making the capsid and the envelope of the vector. Other components are the enzymes involved in the replication of the vector RNA or DNA. Such enzymes can be also involved in the synthesis, maturation or transport of the virus RNA. These enzymes can also be involved in the processing and maturation of viral components, as well as in the integration of the genome of the virus into the cell chromosomes. Enzymes that are components of the viral vectors can also be involved in the reverse transcription of the virus genomic RNA into DNA. Other components of the vector can be protein or peptide that regulate the replication, transcription, transport or translation of the genes or gene products of the viral vector. Such factors can also activate or decrease the expression of cellular genes and they can modulate the defense mechanism of the cells against viruses.

Several viral vectors are well known in the art including adenovirus, adenoviral associated virus (AAV), lentivirus, retrovirus, and herpes simplex virus 1. Accordingly, in an embodiment, the viral vector is a lentivirus, adenovirus, adenoviral associated virus (AAV), retrovirus, or herpes simplex virus 1 vector. Optionally, the viral vector is an AAV vector.

AAV is particularly useful for gene therapy applications as it elicits a limited immune response, exhibits a wide variety of serotypes, and has a stable expression profile. Accordingly, in an embodiment, the viral vector is an AAV vector or a derivative thereof. Optionally, the AAV vector is selected from the group consisting of AAV1, AAV2, AAV5, AAV6, AAV7, AAV8, AAV9, AAVr10 or a derivative thereof.

The term “AAV derivative” as used herein describes a recombinant AAV produced by combining AAV helper plasmids from different AAV serotypes to produce AAV capsids with the combined advantages of more than one serotype. An AAV derivative may further refer to a shuffled AAV derivative which used herein describes an AAV virus containing mutations produced through directed evolutionary or related recombination techniques including but not limited to DNA shuffling. The term “AAV derivative” may also refer to a capsid-modified AAV that can be produced by pseudo typing the sequences of two or more AAV serotypes producing an AAV vector combining characteristics of the two or more serotypes. In an embodiment, the AAV vector is a chimeric, shuffled or capsid modified derivative of AAV.

### Compositions and Kits

In one embodiment there is provided a pharmaceutical composition comprising a nucleic acid construct or viral vector described herein or a derivative of it, and a pharmaceutically acceptable carrier or diluent. The composition may be formulated for use or prepared for administration to a subject using pharmaceutically acceptable formulations known in the art including liposomes or lipid or polymer nanoparticles. Conventional procedures and ingredients for the selection and preparation of suitable formulations are described, for example, in Remington's Pharmaceutical Sciences (2003—20^{th} edition) and in The United States Pharmacopeia: The National Formulary (USP 24 NF19) published in 1999. The term “pharmaceutically acceptable” means compatible with the treatment of animals, in particular, humans.

On this basis, the pharmaceutical compositions could include an active compound or substance, such as a nucleic acid construct or viral vector described herein, in association with one or more pharmaceutically acceptable vehicles or diluents, and contained in buffered solutions with a suitable pH and isosmotic with the physiological fluids. The methods of combining viral vectors the vehicles or combining them with diluents is well known to those skilled in the art. The composition could include a targeting agent for the delivery or transport of the active compound to specified sites within the body, organ, tissue, or cell.

As used herein, the term “diluent” refers to a pharmaceutically acceptable carrier which does not inhibit a physiological activity or property of an active compound, such as lipoxin or a lipoxin analogue, to be administered and does not irritate the subject and does not abrogate the biological activity and properties of the administered compound. Diluents include any and all solvents, dispersion media, coatings, surfactants, antioxidants, preservative salts, preservatives, binders, excipients, disintegration agents, lubricants, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions, formulations, dosages, etc. described herein can be administered for example, by parenteral, intravenous, intracerebroventricular, intrathecal, subcutaneous, or intramuscular administration in dosage unit formulations containing conventional non-toxic pharmaceutically acceptable carriers, adjuvants and vehicles.

The nucleic acid constructs or viral vectors described herein are suitably formulated in a conventional manner into compositions using one or more carriers or diluents. Accordingly, the present description also includes a composition comprising one or more nucleic acid constructs or viral vectors described herein and a carrier or diluent. The nucleic acid constructs or viral vectors described herein are suitably formulated into pharmaceutical compositions for administration to subjects in a biologically compatible form suitable for administration in vivo. Accordingly, the present description further includes a pharmaceutical composition comprising the nucleic acid constructs or viral vectors described herein, and a pharmaceutically acceptable carrier. In some embodiments the pharmaceutical compositions are used in the treatment of any of the diseases, disorders or conditions described herein. In an embodiment, the disease, disorder, or condition is CCDS1.

In some embodiments, the nucleic acid constructs or viral vectors described herein are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, nucleic acid constructs or viral vectors described herein are suitably in a sterile powder form for reconstitution with a suitable vehicle, e.g., sterile pyrogen-free water, before use.

Also provided are kits comprising a nucleic acid construct, viral vector, or pharmaceutical composition as described herein, along with suitable container or packaging and/or instructions for the use thereof, such as for the treatment of CCDS1 in a subject.

### III. METHODS AND USES

As described in the Examples, scAAV9.JeT.SLC6A8 can effectively improve cerebral creatine levels in treated cellular and murine models of cerebral creatine transporter deficiency (CCDS1). Accordingly, one aspect of the disclosure is a method of treating or preventing CCDS1 in a subject in need thereof, comprising administering a therapeutically effective amount of a nucleic acid construct or viral vector disclosed herein to the subject. Another aspect of the disclosure includes use of a nucleic acid construct or viral vector described herein to treat CCDS1. An aspect also includes use of a nucleic acid construct or viral vector described herein in the manufacture of a medicament for treating CCDS1. An aspect also includes a nucleic acid construct or viral vector described herein for use in treating CCDS1.

In an embodiment, the method of treating or preventing CCDS1 comprises administering the therapeutically effective amount of a vector disclosed herein by intravenous, intracerebroventricular or intrathecal injection.

The term cerebral creatine transporter deficiency (CCDS1) describes an X-linked genetic disorder. CCDS1 is caused by loss of function in mutations to SLC6A8 which result in the inability for cells to uptake creatine from the periphery leading to severely depleted or absence of creatine in the central nervous system (CNS). CCDS1 manifests during early childhood and is characterized by a wide range of symptoms including, but not limited to, seizures, intellectual disability, developmental delays and motor function impairment.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. Treatment methods and uses comprise administering to a subject a therapeutically effective amount of a nucleic acid construct or viral vector described herein and optionally consists of a single administration or use, or alternatively comprises a series of administrations or uses.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “subject” as used herein includes all members of the animal kingdom including mammals, and suitably refers to humans. Optionally, the term “subject” includes mammals that have been diagnosed with CCDS1. In an embodiment, the subject is a mammal. In another embodiment, the subject is human. In one embodiment, the term “subject” refers to a human having, or suspected of having, CCDS1.

The term “subject in need thereof” refers to a subject that could benefit from the method(s) or treatment(s) described herein, and optionally refers to a subject with CCDS1, or optionally a subject with increased risk of CCDS1, such as a subject with a strong genetic predisposition.

The term “administered” or “administering” as used herein means administration of a therapeutically effective amount of a compound or composition of the disclosure to a cell either in cell culture or in a subject. The nucleic acid constructs or viral vectors described herein may be administered to a subject in a variety of forms depending on the selected route of administration, as will be understood by those skilled in the art. For example, the nucleic acid constructs or viral vectors described herein may be administered by parenteral administration or direct injection into brain and the pharmaceutical compositions formulated accordingly. In some embodiments, administration is by means of a pump for periodic or continuous delivery.

The nucleic acid constructs or viral vectors described herein may be administered to a subject in a variety of forms depending on the selected route of administration, as will be understood by those skilled in the art. For example, the nucleic acid constructs or viral vectors described herein may be administered by parenteral administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, intrapulmonary (for example, by use of an aerosol), and intrathecal modes of administration. Parenteral administration may be by continuous infusion over a selected period of time.

As used herein, the phrase “intrathecal” means existing or taking place within, or administered into the fluid-filled space between the thin layers of tissue that cover the brain and spinal cord.

As used herein, the phrase “intravenous” means existing or taking place within, or administered into, a vein or veins. Intravenous delivery of gene therapy vectors allows for widespread delivery and transduction to organs and tissues in a subject.

As used herein, the phrase “effective amount” or “therapeutically effective amount” means an amount effective, at dosages and for periods of time necessary to achieve the desired result. For example, in the context of treating CCDS1, an effective amount is an amount that for example increases the level of creatine in the brain compared to the response obtained without administration of the compound. Effective amounts may vary according to factors such as the disease state, age, sex, and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

Suitable administration schedules may include, without limitation, at least once a week, from about once in lifetime, one time per two weeks, three weeks or one month, about one time per week to about once daily. The length of the treatment period may depend on a variety of factors, such as the severity of the disease, disorder or condition, the age of the subject, the concentration and/or the activity of the nucleic acid constructs or viral vectors described herein. It will also be appreciated that the effective dosage of the nucleic acid constructs or viral vectors described herein used for the treatment may increase or decrease over the course of a particular treatment regime. Changes in dosage may result and become apparent by standard diagnostic assays known in the art. In some instances, chronic administration is required. For example, the nucleic acid construct or viral vector described herein are administered to the subject in an amount and for duration sufficient to treat the subject.

### EXAMPLES

The following non-limiting examples are illustrative of the present disclosure:

### Example 1: Materials and Methods

### Animal Models

B6(Cg)Jmice with a deletion of Exon 2-4 of SLC6A8 were obtained from Jackson laboratories (B6(Cg)-Slc6a8^{tm1.2Clar}/J). As SLC6A8 is an X linked gene all experimental animals were male mice produced from a heterozygous female bred to a WT male mouse (SLC6A8 Ko male mice are infertile). All mice were kept with a 12-hour photoperiod with the lights on beginning at 7 am and all interactions with the mice were done during this time. All procedures and cohort sizes have been approved by the Queen's University Animal Care Committee according to the guidance of the Canadian Council on Animal Care.

### ICV Injections

Protocol was adapted from Kim et al 2014²⁹. In short P0 pups were anesthetized on ice and injected with 0.5 ul of scAAV9.JeT.SLC6A8 in each hemisphere.

### IT Injections

IT injections were performed at 6 weeks of age. Mice were anesthetized under isoflurane before 10 ul scAAV9.JeT.SLC6A8 or vehicle was administered into the spinal cord between vertebrate L5 and L6.

### Statistical Analysis

All were performed using GraphPad Prism™ 10. A Shapiro Wilk normality test was used before all tests to assess data normality. A Brown-Forsythe one test followed by a dunnet T3 test was performed if brown-forcyth ANOVA detected any significant differences. Significance threshold before multiple corrections for all experiments was set to P<0.05.

### Cell Culture and Transfection

Patient fibroblasts were obtained from Cornell (GM27448, Coriell Institute) and immortalized using lenti system (G256, Applied Biological Materials Inc). Cells were grown in creatine free IMDM media (12440053, Gibco), with penicillin streptomycin (P4333, Sigma) and dialyzed FBS (F1051, Sigma) which was created in short via dialyzing the FBS 20:1 in 4 °C 0.15 mM NaCl for 1 hour 4× through a 11 kDA membrane.

Immortalized cells were cultured in Iscove's Modified Dulbecco's Medium (Sigma 13390) with 10% dFBS (Wisent 080-450, dialyzed as described above) and 1% Penicillin-Streptomycin (Sigma P4333). 750 k cells were plated per well in a 6 cm well plate 48 hours before transfection occurred, and cells were transfected with 2.2 μg of plasmid DNA per well using the JeT Optimus transfection reagent (Polyplus 117) according to manufacturer protocols. Cells were harvested 72 hours post transfection for metabolite analysis by mass spectrometry, 1 hour before harvest cells were incubated with 100 uM creatine (C0780 Sigma).

qPCR

All qPCR was performed in technical triplicates using the BioRad CFX96™ Touch Real-Time PCR Detection System and PowerUp™ SYBR™ Green Master mix (Thermofisher, A25741). DNA isolation was performed using gSYNC™ DNA Extraction Kit (Geneaid, GS100) according to manufacturer protocol and stored at −20 °C until analysis. All primers were validated according to MIQE guidelines.

### Copy Number Variance Analysis

Copy number is reported as the mean relative number of codon optimized double stranded SLC6A8 molecules per molecule of laminB2 molecules.

LaminB2 primers were used as the reference gene for copy number analysis. Reference genes were selected for melting temperature and primer efficiency according to MIQE guidelines. SLC6A8 primers were utilized for both gene expression and copy number analysis. Primer sequences are as shown in Table 1.

TABLE 1
Mass spectrometry quantitation of creatine
metabolites

| | | SEQ |
|---|---|---|
| Primer | Sequence (5′-3′) | ID NO |
| LaminB2 Forward | GGACCCAAGGACTACCTCAAGGG | 6 |
| LaminB2 Reverse | AGGGCACCTCCATCTCGGAAAC | 7 |
| Codon optimized | CAGCCTGGGGCAGTTTATGA | 8 |
| SLC6A8 Forward | | |
| Codon optimized | ACAATGACCATGCTGGCGTA | 9 |
| SLC6A8 Reverse | | |

Mass spectrometry (MS) analysis was performed as described in Khoja et al.,³⁰ to measure creatine, arginine, guanidinoacetate and creatinine (creatine metabolites). In short tissues were harvested and flash frozen. Tissues were then weighed and digested in a bead rotor before protein precipitation via 30% TCA VWR International (Radnor, PA). Samples were then mixed with an internal standard and methanol before solvent evaporation. Samples were then suspended in butanol before evaporation and finally resuspended in methanol before mass spectrometry analysis. HPLC separation was done by the ExionLC™ AD UHPLC system and mass spectrometry was run on a QTRAP™ 6500plus (AB Sciex, Framingham, MA). Data processing and quantification was performed using Analyst 1.7.0 software (AB Sciex).

### Viral Vector Preparation

Single stranded AAV9 vector preparation was performed in HEK293 cells at UNC vector core. The vector construct contained the small synthetic JeT promoter and codon optimized SLC6A8 coding sequence (NM_001142806.1) with a synthetic SV40 poly-A tail. Viral vector was resuspended in PBS with 5% D-Sorbitol and diluted so that each treatment group received an equal volume during ICM injections.

### Grip Strength

To assess the grip strength mice at 13 weeks of age were tasked with holding a weight for up to three seconds (1 second per point) and given a progressively heavier weight until they failed three times. Mice were held by the tail and weights were attached to steel wool where mice were allowed to grip with their forepaws only. Up to five weights were used for a maximum score of fifteen points.

### Example 2: An In Vitro Study Transfection of SLC6A8 Induced Creatine Transport in Patient Fibroblasts

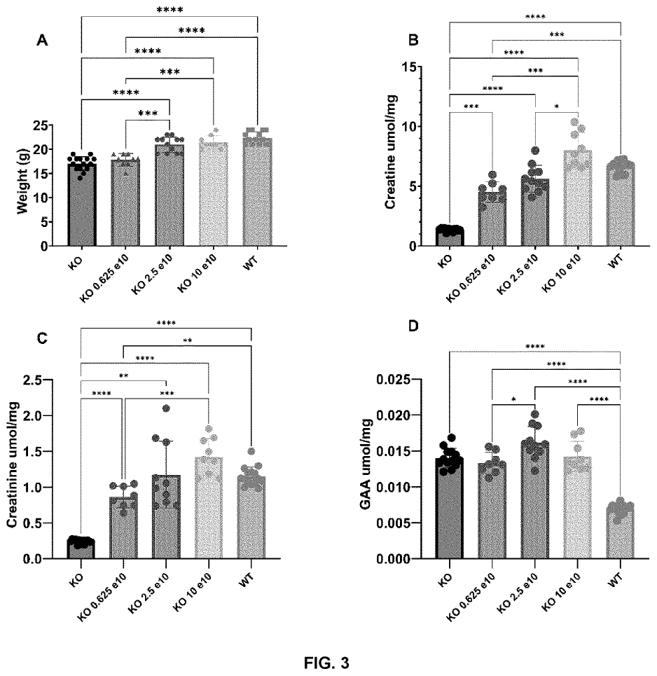

To confirm the efficacy of the codon optimized SLC6A8 and the JeT promoter a plasmid containing the scAAV9SLC6A8 construct was tested for the ability to restore creatine transport in SLC6A8 KO patient fibroblasts. To remove any residual creatine the patient cells were grown in creatine free media for 14 days before transfection. 72-hours post transfection cells were incubated in media with (XMolar) creatine for 1 hour before harvesting and metabolite quantification by mass spectrometry. Intracellular creatine and creatinine were over 30-fold greater compared to GFP transfected controls (FIG. 2.)

### Example 3: Intracerebroventricular Administration of scAAV9SLC6A8 Increased Brain Creatine in a Dose Dependent Manner

Intracerebroventricular (ICV) allows for direct delivery to the mouse brain at postnatal day 0 (P0), this allows for both efficient AAV delivery as well as gene expression at an early developmental stage. ICV injections were performed at P0 with a dosage of between 0.625e10 to 1.0e10 vg of scAA V9.JeT.SLC6A8 or vehicle solution. 5-weeks post injection mice were sacrificed with organs flash frozen for mass spectrometry quantification of creatine metabolites. Doses of 2.5e10 and 10e10 vector genomes (vg) both resulted in a statistically significant increase in weight relative to vehicle injected KO mice and a dose of 10e10 vg was not statistically significant from WT mice (FIG. 3A). Both creatine and creatinine content were vastly increased in the midsection of the brain in all doses, the 10e10 vg dose had a higher concentration of creatine than the WT although not statistically significant (FIG. 3B, C). The 10e10 vg dose did not appear to have reached the maximum effective dose with respect to brain creatine response (FIG. 3). The viral vector did not decrease the GAA concentration in the midsection of the brain however, this concentration was 100-fold lower than observed in GAMT deficiency and unlikely to be clinically relevant (FIG. 3).

### Example 4: Intracerebroventricular Administration of scAAV9SLC6A8 Improved Body Mass and Reduced Hyperactivity 6 Months Following ICV Administration

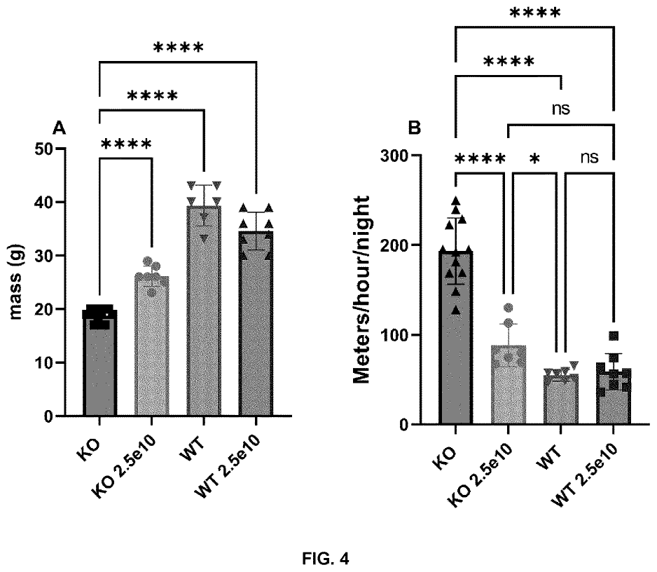

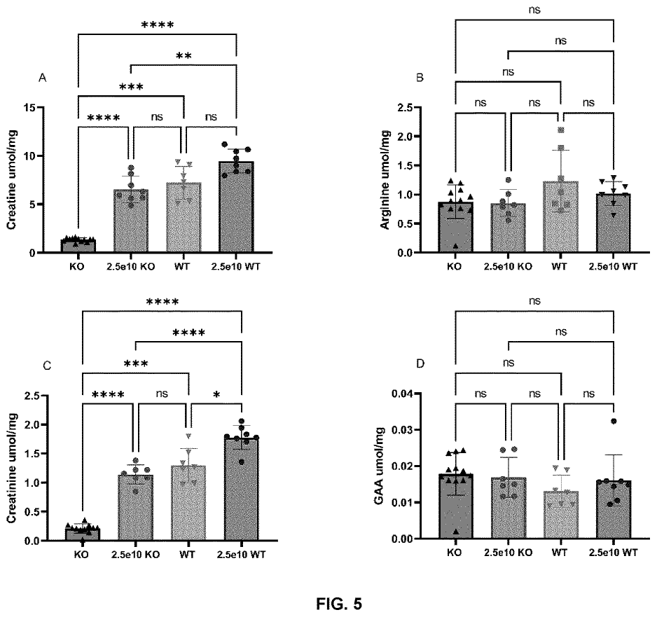

ICV injections were performed at P0 with a dosage of 2.5e10 vg of scAAV9.JeT.SLC6A8. 5 weeks post injection mice were started on a rapamycin and prednisone immunosuppression regimen³¹. Mice treated with 2.5e10 vg of scAAV9.JeT.SLC6A8 showed a statistically significant increase in body mass 6 months post injection. SLC6A8 mutations lead to a variety of behavioral disorders including hyperactivity in both mice and humans. Distance traveled during the dark period over 14 days was measured using the Tecniplast DVC™. The first 4 days were excluded to allow for the mice to adjust to the new environment and then the average movement period per hour during the dark phase was calculated for each mouse. Treated mice had drastically reduced hyperactivity compared to KO controls, although treated mice still traveled significantly more than WT mice (FIG. 4B).

### Example 5: Intracerebroventricular Administration of scAAV9SLC6A8 Restored Creatine in the Midsection of the Brain 6 Months Post Injection

ICV injections were performed at P0 with a dosage of 2.5e10 vg of scAAV9.JeT.SLC6A8. 5 weeks post injection mice were started on a rapamycin and prednisone immunosuppression regimen. Mice were euthanized at 24 weeks of age for mass spectrometry analysis. In the midsection in the brain, there was a statistically significant 5-fold elevation of both creatine and creatinine in treated mice relative to KO mice (FIG. 5). WT treated mice showed elevated creatine and creatinine relative to the untreated WT mice although the difference was not statistically significant for creatine (FIG. 5). Both arginine and GAA remained constant with no significant difference between any groups in the brain midsection (FIG. 5).

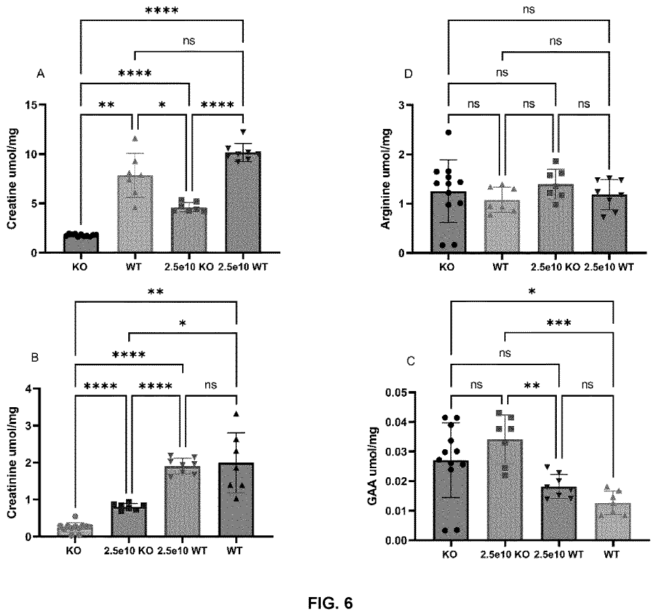

In the caudal section in the brain there was a statistically significant 5-fold elevation of both creatine and creatinine in treated mice relative to KO mice. Unlike in the midsection of the brain there was no statistically significant differences between WT treated and untreated mice likely due to a reduced transduction in the caudal section relative to the midsection (FIG. 6). GAA was also elevated in both treated and untreated KO mice relative to WT mice. While statistically significant this likely had minimal impact on brain function as while GAA is thought to be the source of toxicity in GAMT creatine deficiencies the elevation seen in these mice was still 100-fold lower than in GAMT deficiency.

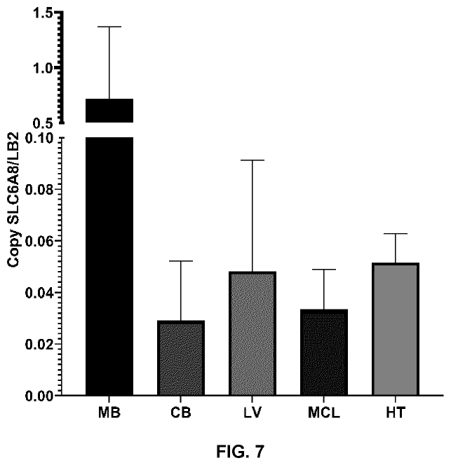

The biodistribution at 24 weeks of age following ICV injections showed a high concentration in the midsection of the brain (FIG. 7). This corresponded to the distance from the injection site.

### Example 6: Intrathecal Administration of scAAV9SLC6A8 Resulted in a Mild Increase in Brain Creatine, Body Mass and Grip Strength 8 Week Following Treatment

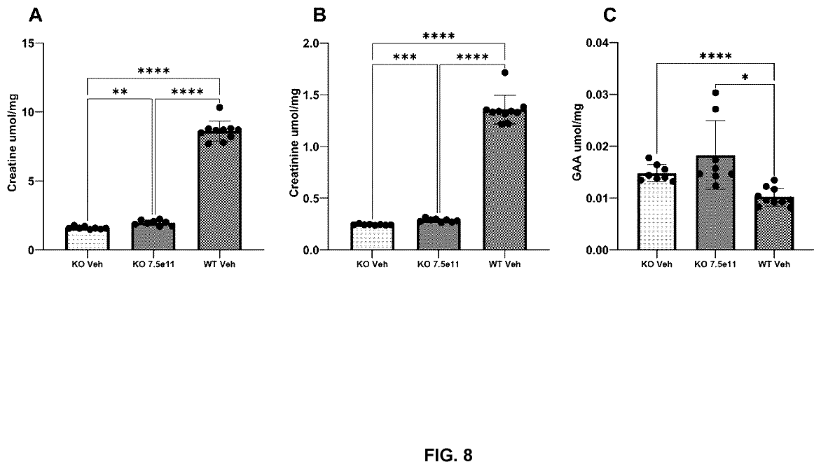

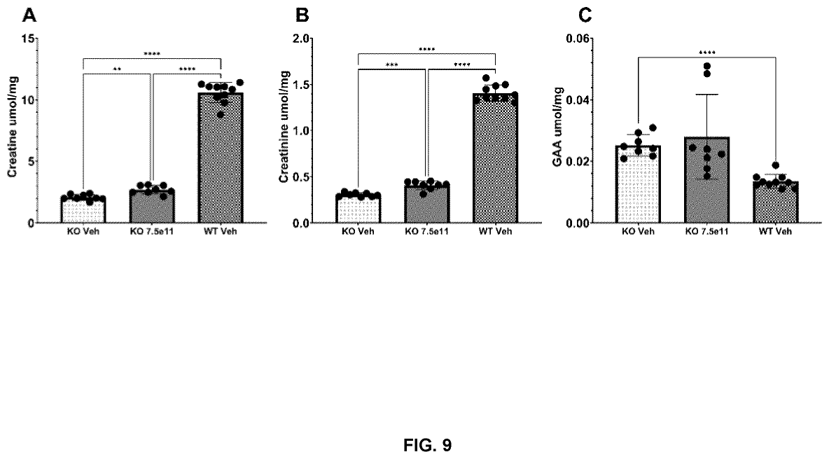

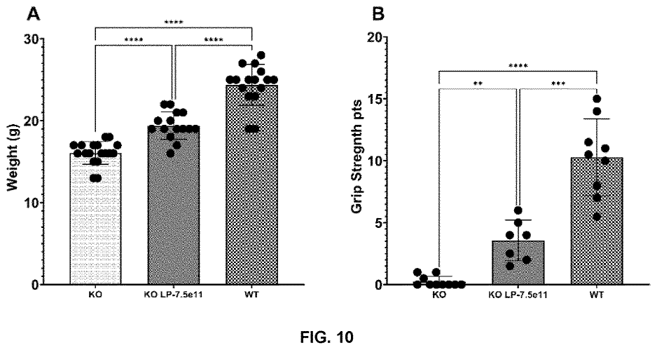

Intrathecal injections were performed at 6 weeks of age with a dosage of 7.5e 11vg of scAAV9.JeT.SLC6A8. Mice were given immunosuppression of rapamycin and prednisone beginning at 5 weeks of age until endpoint. At 13 weeks of age or 8 weeks post injection mice were euthanized and the brain was harvested for mass spectrometry analysis. Brain creatine and creatinine were elevated in both the midsection and caudal section of the brain (FIGS. 8-9). GAA in contrast appeared to be slightly elevated in both KO mice. While statistically significant the increase in creatine in both the caudal and mid section of the brain were mild compared to intracerebroventricular injections (FIGS. 8-9, 3). Yet even with a mild increase in brain creatine there were substantial increases in both mouse body mass and improved grip strength indicating that there may be symptom improvement without full creatine restoration in the brain (FIG. 10).

### Example 7: Intrathecal Administration of scAAV9-SLC6A8 Resulted in a Mild Increase in Brain Creatine, Body Mass and Grip Strength 18 Weeks Following Treatment

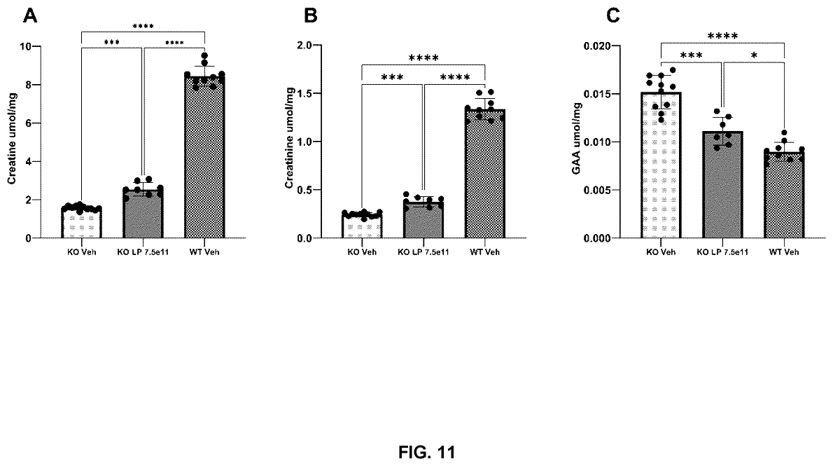

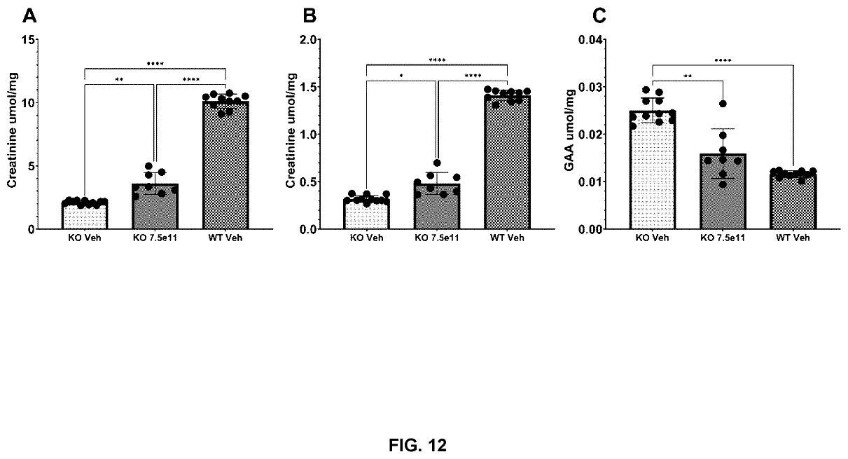

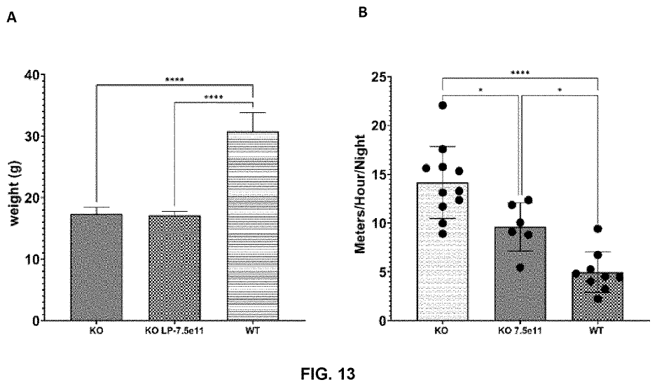

Intrathecal injections were performed at 6 weeks of age with a dosage of 7.5e 11 vg of scAAV9.JeT.SLC6A8. Mice were started on a rapamycin and prednisone immunosuppression at 5 weeks of age according to a previously described regimen³¹. At 24 weeks of age or 18 weeks post injection mice were euthanized and the brain was harvested for mass spectrometry analysis. Brain creatine and creatinine were elevated while GAA was decreased in both the midsection and caudal section of the brain following treatment (FIG. 11,12). The increase in brain creatine appeared to be greater at the 24-week endpoint compared to the 13-week endpoint, although still substantially below WT or ICV treated mice (FIGS. 11-12, 9-10, 3,). This mild increase in creatine again resulted in a significant and potentially clinically relevant decrease in overnight hyperactivity further supporting evidence that there may be patient benefit without full creatine restoration (FIG. 13).

While the present disclosure has been described with reference to examples, it is to be understood that the scope of the claims should not be limited by the embodiments set forth in the examples but should be given the broadest interpretation consistent with the description as a whole.

### SEQUENCES

SEQ ID NO 1: SLC6A8 codon optimized
ATGGCCAAGAAGTCCGCCGAGAACGGCATCTACTCCGTGAGCGGGGATGA
GAAGAAGGGGCCCCTGATCGCCCCCGGCCCCGATGGCGCCCCCGCCAAGG
GGGACGGCCCCGTGGGGCTCGGGACACCCGGAGGGAGACTGGCCGTGCCC
CCTAGGGAGACATGGACACGGCAGATGGATTTTATCATGAGCTGCGTGGG
CTTCGCCGTGGGGCTGGGGAACGTGTGGAGGTTTCCCTACCTGTGCTACA
AGAACGGCGGCGGCGTGTTCCTGATCCCCTACGTGCTGATCGCCCTGGTG
GGCGGCATCCCCATCTTCTTCCTGGAAATCAGCCTGGGGCAGTTTATGAA
GGCTGGGAGTATCAACGTGTGGAACATCTGCCCATTGTTCAAGGGCCTGG
GCTACGCCAGCATGGTCATTGTGTTCTACTGCAACACCTACTACATCATG
GTGCTGGCCTGGGGGTTCTACTACCTGGTCAAGAGCTTCACAACAACCCT
GCCATGGGCCACATGCGGCCACACATGGAACACACCTGACTGCGTCGAGA
TCTTCCGGCACGAGGATTGCGCCAACGCCAGCCTGGCCAACCTGACCTGC
GATCAGCTGGCCGACAGGAGGAGCCCCGTGATCGAGTTCTGGGAGAACAA
GGTGCTGCGGCTGTCCGGCGGGCTGGAGGTGCCCGGCGCCCTGAACTGGG
AGGTGACCCTCTGCCTGCTGGCCTGCTGGGTGCTGGTGTACTTTTGCGTG
TGGAAGGGGGTGAAGTCCACCGGCAAGATCGTGTACTTCACCGCCACATT
CCCTTACGTGGTGCTGGTGGTCCTGCTGGTCAGGGGCGTGCTCCTGCCCG
GAGCCCTGGACGGCATCATCTACTACCTGAAGCCCGATTGGAGCAAGCTG
GGGTCCCCCCAGGTGTGGATCGATGCCGGGACACAGATCTTCTTTTCCTA
CGCCATTGGACTCGGCGCCCTGACAGCCCTGGGAAGTTACAACAGGTTTA
ACAACAACTGCTACAAGGATGCCATCATTTTGGCTCTGATTAACAGCGGA
ACTAGCTTCTTTGCAGGGTTCGTCGTGTTTAGCATTCTGGGCTTCATGGC
CGCTGAACAGGGAGTGCACATTTCCAAGGTGGCTGAATCCGGCCCCGGCC
TGGCCTTCATCGCCTACCCCAGGGCCGTGACACTGATGCCCGTGGCCCCC
CTGTGGGCCGCCCTGTTCTTCTTCATGCTGCTGCTGCTGGGCCTGGACAG
CCAGTTCGTGGGGGTGGAGGGGTTCATCACAGGCCTGCTGGATCTGCTGC
CAGCCAGCTACTACTTCCGGTTCCAGCGGGAGATCAGCGTGGCCCTGTGC
TGCGCCCTGTGCTTCGTGATCGATCTGAGCATGGTGACCGACGGGGGGAT
GTACGTGTTCCAGCTGTTCGACTACTACAGCGCCTCCGGGACAACCCTGC
TGTGGCAGGCCTTCTGGGAGTGCGTGGTGGTGGCCTGGGTGTACGGGGCC
GACAGGTTCATGGATGATATTGCCTGCATGATCGGCTACCGGCCCTGCCC
CTGGATGAAGTGGTGCTGGAGCTTCTTCACACCCCTGGTGTGCATGGGCA
TCTTCATCTTCAACGTGGTGTACTACGAGCCCCTGGTGTACAACAACACC
TACGTGTACCCATGGTGGGGGGAGGCCATGGGCTGGGCCTTCGCCCTGAG
CAGCATGCTGTGCGTGCCCCTGCACCTGCTGGGGTGCCTGCTGAGGGCCA
AGGGGACAATGGCCGAGCGGTGGCAGCACCTGACACAGCCCATCTGGGGG
CTGCACCACCTGGAGTACAGGGCCCAGGATGCCGACGTGAGGGGCCTGAC
AACCCTGACCCCAGTGAGTGAATCTAGTAAGGTAGTAGTAGTTGAGTCAG
TGATGTGA
SEQ ID NO 2: Full Viral vector sequence
GGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGA
GCGCGCAGAGAGGGAGTGGCCAACTCCATCACTAGGGGTTCCTGGAGGGG
TGGAGTCGTGAGCAGAATTCGCCGGGCGGAGTTAGGGCGGAGCCAATCAG
CGTGCGCCGTTCCGAAAGTTGCCTTTTATGGCTGGGCGGAGAATGGGCGG
TGAACGCCGATGATTATATAAGGACGCGCCGGGTGTGGCACAGCTAGTTC
CGTCGCAGCCGGGATTTGGGTCGCGGTTCTTGTTTGTTCCGGAAAGCCAC
CATGGCCAAGAAGTCCGCCGAGAACGGCATCTACTCCGTGAGCGGGGATG
AGAAGAAGGGGCCCCTGATCGCCCCCGGCCCCGATGGCGCCCCCGCCAAG
GGGGACGGCCCCGTGGGGCTCGGGACACCCGGAGGGAGACTGGCCGTGCC
CCCTAGGGAGACATGGACACGGCAGATGGATTTTATCATGAGCTGCGTGG
GCTTCGCCGTGGGGCTGGGGAACGTGTGGAGGTTTCCCTACCTGTGCTAC
AAGAACGGCGGCGGCGTGTTCCTGATCCCCTACGTGCTGATCGCCCTGGT
GGGCGGCATCCCCATCTTCTTCCTGGAAATCAGCCTGGGGCAGTTTATGA
AGGCTGGGAGTATCAACGTGTGGAACATCTGCCCATTGTTCAAGGGCCTG
GGCTACGCCAGCATGGTCATTGTGTTCTACTGCAACACCTACTACATCAT
GGTGCTGGCCTGGGGGTTCTACTACCTGGTCAAGAGCTTCACAACAACCC
TGCCATGGGCCACATGCGGCCACACATGGAACACACCTGACTGCGTCGAG
ATCGAGTTCTGGGAGAACAAGGTGCTGCGGCTGTCCGGGGGGCTGGAGGT
ATCTTCCGGCACGAGGATTGCGCCAACGCCAGCCTGGCCAACCTGACCTG
CGATCAGCTGGCCGACAGGAGGAGCCCCGTGGCCCGGCGCCCTGAACTGG
GAGGTGACCCTCTGCCTGCTGGCCTGCTGGGTGCTGGTGTACTTTTGCGT
GTGGAAGGGGGTGAAGTCCACCGGCAAGATCGTGTACTTCACCGCCACAT
TCCCTTACGTGGTGCTGGTGGTCCTGCTGGTCAGGGGCGTGCTCCTGCCC
GGAGCCCTGGACGGCATCATCTACTACCTGAAGCCCGATTGGAGCAAGCT
GGGGTCCCCCCAGGTGTGGATCGATGCCGGGACACAGATCTTCTTTTCCT
ACGCCATTGGACTCGGCGCCCTGACAGCCCTGGGAAGTTACAACAGGTTT
AACAACAACTGCTACAAGGATGCCATCATTTTGGCTCTGATTAACAGCGG
AACTAGCTTCTTTGCAGGGTTCGTCGTGTTTAGCATTCTGGGCTTCATGG
CCGCTGAACAGGGAGTGCACATTTCCAAGGTGGCTGAATCCGGCCCCGGC
CTGGCCTTCATCGCCTACCCCAGGGCCGTGACACTGATGCCCGTGGCCCC
CCTGTGGGCCGCCCTGTTCTTCTTCATGCTGCTGCTGCTGGGCCTGGACA
GCCAGTTCGTGGGGGTGGAGGGGTTCATCACAGGCCTGCTGGATCTGCTG
CCAGCCAGCTACTACTTCCGGTTCCAGCGGGAGATCAGCGTGGCCCTGTG
ATGGATGATATTGCCTGCATGATCGGCTACCGGCCCTGCCCCTGGATGAA
CTGCGCCCTGTGCTTCGTGATCGATCTGAGCATGGTGACCGACGGGGGGA
TGTACGTGTTCCAGCTGTTCGACTACTACAGCGCCTCCGGGACAACCCTG
CTGTGGCAGGCCTTCTGGGAGTGCGTGGTGGTGGCCTGGGTGTACGGGGC
CGACAGGTTCGTGGTGCTGGAGCTTCTTCACACCCCTGGTGTGCATGGGC
ATCTTCATCTTCAACGTGGTGTACTACGAGCCCCTGGTGTACAACAACAC
CTACGTGTACCCATGGTGGGGGGAGGCCATGGGCTGGGCCTTCGCCCTGA
GCAGCATGCTGTGCGTGCCCCTGCACCTGCTGGGGTGCCTGCTGAGGGCC
AAGGGGACAATGGCCGAGCGGTGGCAGCACCTGACACAGCCCATCTGGGG
GCTGCACCACCTGGAGTACAGGGCCCAGGATGCCGACGTGAGGGGCCTGA
CAACCCTGACCCCAGTGAGTGAATCTAGTAAGGTAGTAGTAGTTGAGTCA
GTGATGTGAGCAGTCGACGCCAATAAAGAGCTCAGATGCATCGATCAGAG
TGTGTTGGTTTTTTGTGTGCCACTCCCTCTCTGCGCGCTCGCTCGCTCAC
TGAGGCCGGGCGACCAAAGGTCGCCCGACGCCCGGGCTTTGCCCGGGGGG
CCTCAGTGAGCGAGCGAGCGCGCAGAGAGGGA
SEQ ID NO 3: 5′ ITR
GGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGA
GCGCGCAGAGAGGGAGTGGCCAACTCCATCACTAGGGGTTCCTGGAGGGG
TGGAGTCGTGAGCAGAATTCGCC
SEQ ID NO 4: JeT Promoter
GGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGG
ACGCGCCGGGTGTGGCACAGCTAGTTCCGTCGCAGCCGGGATTTGGGTCG
CGGTTCTTGTTTGTTCCGGAAAGCCACC
SEQ ID NO 5: 3′ ITR
GCAGTCGACGCCAATAAAGAGCTCAGATGCATCGATCAGAGTGTGTTGGT
TTTTTGTGTGCCACTCCCTCTCTGCGCGCTCGCTCGCTCACTGAGGCCGG
GCGACCAAAGGTCGCCCGACGCCCGGGCTTTGCCCGGGCGGCCTCAGTGA
GCGAGCGAGCGCGCAGAGAGGGA

### REFERENCES

1. Item, C. B. et al. Arginine:Glycine Amidinotransferase Deficiency: The Third Inborn Error of Creatine Metabolism in Humans. Am. J. Hum. Genet. 69, 1127-1133 (2001).
2. Stöckler, S., Isbrandt, D., Hanefeld, F., Schmidt, B. & von Figura, K. Guanidinoacetate methyltransferase deficiency: the first inborn error of creatine metabolism in man. Am. J. Hum. Genet. 58, 914-922 (1996).
3. Hanna-El-Daher, L., Beard, E., Henry, H., Tenenbaum, L. & Braissant, O. Mild guanidinoacetate increase under partial guanidinoacetate methyltransferase deficiency strongly affects brain cell development. Neurobiol. Dis. 79, 14-27 (2015).
4. Gregor, P., Nash, S. R., Caron, M. G., Seldin, M. F. & Warren, S. T. Assignment of the creatine transporter gene (SLC6A8) to human chromosome Xq28 telomeric to G6PD. Genomics 25, 332-333 (1995).
5. Salomons, G. S. et al. X-Linked Creatine-Transporter Gene (SLC6A8) Defect: A New Creatine-Deficiency Syndrome. Am. J. Hum. Genet. 68, 1497-1500 (2001).
6. Sora, I. et al. The Cloning and Expression of a Human Creatine Transporter. Biochem. Biophys. Res. Commun. 204, 419-427 (1994).
7. Iyer, G. S. et al. Identification of a Testis-Expressed Creatine Transporter Gene at 16p11.2 and Confirmation of the X-Linked Locus to Xq28. Genomics 34, 143-146 (1996).
8. Bayou, N. et al. The Creatine Transporter Gene Paralogous at 16p11.2 Is Expressed in Human Brain. Comp. Funct. Genomics 2008, e609684 (2008).
9. Wallimann, T., Tokarska-Schlattner, M. & Schlattner, U. The creatine kinase system and pleiotropic effects of creatine. Amino Acids 40, 1271-1296 (2011).
10. Braissant, O. Creatine and guanidinoacetate transport at blood-brain and blood-cerebrospinal fluid barriers. J. Inherit. Metab. Dis. 35, 655-664 (2012).
11. Persky, A. M., Brazeau, G. A. & Hochhaus, G. Pharmacokinetics of the Dietary Supplement Creatine. Clin. Pharmacokinet. 42, 557-574 (2003).
12. Nasrallah, F., Feki, M. & Kaabachi, N. Creatine and Creatine Deficiency Syndromes: Biochemical and Clinical Aspects. Pediatr. Neurol. 42, 163-171 (2010).
13. Braissant, O., Béard, E., Torrent, C. & Henry, H. Dissociation of AGAT, GAMT and SLC6A8 in CNS: relevance to creatine deficiency syndromes. Neurobiol. Dis. 37, 423-433 (2010).
14. Perna, M. K. et al. Creatine transporter deficiency leads to increased whole body and cellular metabolism. Amino Acids 48, 2057-2065 (2016).
15. van de Kamp, J. M., Mancini, G. M. & Salomons, G. S. X-linked creatine transporter deficiency: clinical aspects and pathophysiology. J. Inherit. Metab. Dis. 37, 715-733 (2014).
16. Clark, A. J. et al. X-linked creatine transporter (SLC6A8) mutations in about 1% of males with mental retardation of unknown etiology. Hum. Genet. 119, 604-610 (2006).
17. Rosenberg, E. H. et al. High Prevalence of SLC6A8 Deficiency in X-Linked Mental Retardation. Am. J. Hum. Genet. 75, 97-105 (2004).
18. Baroncelli, L. et al. A novel mouse model of creatine transporter deficiency. F1000Research 3, 228 (2014).
19. Skelton, M. R. et al. Creatine Transporter (CrT; Slc6a8) Knockout Mice as a Model of Human CrT Deficiency. PLoS ONE 6, e16187 (2011).
20. Hanna-El-Daher, L. & Braissant, O. Creatine synthesis and exchanges between brain cells: What can be learned from human creatine deficiencies and various experimental models? Amino Acids 48, 1877-1895 (2016).
21. Abdulla, Z. I. et al. Deletion of the Creatine Transporter (Slc6a8) in Dopaminergic Neurons Leads to Hyperactivity in Mice. J. Mol. Neurosci. 70, 102-111 (2020).
22. Duran-Trio, L. et al. A new rat model of creatine transporter deficiency reveals behavioral disorder and altered brain metabolism. Sci. Rep. 11, 1636 (2021).
23. Udobi, K. C. et al. Deletion of the creatine transporter gene in neonatal, but not adult, mice lead to cognitive deficits. http://biorxiv.org/lookup/doi/10.1101/582320 (2019) doi:10.1101/582320.
24. Kuzmin, D. A. et al. The clinical landscape for AAV gene therapies. Nat. Rev. Drug Discov. 20, 173-174 (2021).
25. Aschauer, D. F., Kreuz, S. & Rumpel, S. Analysis of Transduction Efficiency, Tropism and Axonal Transport of AAV Serotypes 1, 2, 5, 6, 8 and 9 in the Mouse Brain. PLOS ONE 8, e76310 (2013).
26. Ferrari, F. K., Samulski, T., Shenk, T. & Samulski, R. J. Second-strand synthesis is a rate-limiting step for efficient transduction by recombinant adeno-associated virus vectors. J. Virol. 70, 3227-3234 (1996).
27. McCarty, D. M. Self-complementary AAV Vectors; Advances and Applications. Mol. Ther. 16, 1648-1656 (2008).
28. Wu, J. et al. Self-Complementary Recombinant Adeno-Associated Viral Vectors: Packaging Capacity And The Role of Rep Proteins in Vector Purity. Hum. Gene Ther. 18, 171-182 (2007).
29. Kim, J.-Y., Grunke, S. D., Levites, Y., Golde, T. E. & Jankowsky, J. L. Intracerebroventricular Viral Injection of the Neonatal Mouse Brain for Persistent and Widespread Neuronal Transduction. JoVE J. Vis. Exp. e51863 (2014) doi:10.3791/51863.
30. Khoja S, et al. Gene therapy for guanidinoacetate methyltransferase deficiency restores cerebral and myocardial creatine while resolving behavioral abnormalities. Mol Ther Methods Clin Dev. 2022 Mar. 28; 25:278-296. doi: 10.1016/j.omtm.2022.03.015. PMID: 35505663; PMCID: PMC9051621.
31. Kot S, et al. Investigating Immune Responses to the scAAV9-HEXM Gene Therapy Treatment in Tay-Sachs Disease and Sandhoff Disease Mouse Models. Int J Mol Sci. 2021 Jun. 23; 22(13):6751. doi: 10.3390/ijms22136751. PMID: 34201771; PMCID: PMC8268035.

1. A nucleic acid construct comprising a nucleotide sequence encoding a Solute Carrier Family 6 Member 8 (SLC6A8) protein operably linked to a promoter and a transcription termination site, wherein the nucleotide sequence is at least 95% identical to the nucleotide sequence of SEQ ID NO:1 and wherein the protein encoded by said nucleic acid sequence retains SLC6A8 activity.

2. The nucleic acid construct of claim 1, wherein the nucleotide sequence is at least 99% identical to the nucleotide sequence of SEQ ID NO: 1, and wherein the protein encoded by said nucleic acid sequence retains SLC6A8 activity.

3. The nucleic acid construct of claim 1, wherein the nucleotide sequence encoding the SLC6A8 protein is set forth in SEQ ID NO: 1.

4. The nucleic acid construct of claim 1, wherein the SLC6A8 protein has an amino acid sequence that is at least 95%, or at least 99% identical to the protein encoded by SEQ ID NO: 1, and which retains SLC6A8 activity.

5. The nucleic acid construct according to claim 1, wherein the promoter is a constitutive promoter or a tissue- or cell-specific promoter.

6. The nucleic acid construct according to claim 5, wherein the promoter is selected from the group consisting of: chicken beta actin (CBA), chicken β-actin hybrid (CBh), CMV early enhancer (CAG), Elongation Factor 1α (eF-1a), simian virus 40 early promoter (SV40), human phosphoglycerate kinase 1 (PGK), cytomegalovirus immediate-early promoter (CMV), human β-actin (hACTB) and JeT synthetic promoter.

7. The nucleic acid construct of claim 1, comprising a sequence set forth in SEQ ID NO: 2.

8. A viral vector comprising the nucleic acid construct according to claim 1.

9. The viral vector according to claim 8, wherein the viral vector is an Adeno-Associated Virus (AAV) vector or a derivative thereof.

10. The viral vector according to claim 9, wherein the AAV vector is selected from the group consisting of: AAV1, AAV2, AAV5, AAV6, AAV7, AAV8, AAV9, AAVrh10 or a derivative thereof.

11. The viral vector according to claim 9, wherein the viral vector is a chimeric, shuffled or capsid modified derivative of AAV.

12. A pharmaceutical composition comprising the nucleic acid construct of claim 1 or a viral vector comprising said nucleic acid construct, and a pharmaceutically acceptable carrier or diluent.

13. The pharmaceutical composition of claim 12, wherein the pharmaceutically acceptable carrier is a lipid nanoparticle or polymer nanoparticle.

14. The pharmaceutical composition of claim 12, wherein the pharmaceutical composition is formulated for intravenous, intracerebroventricular or intrathecal administration.

15. A method of treating or preventing CCDS1 in a subject in need thereof, the method comprising administering the viral vector according to claim 8 or a pharmaceutical composition comprising said viral vector to the subject in need thereof.

16. (canceled)

17. The method of claim 15, wherein the viral vector or pharmaceutical composition is formulated for intravenous, intracerebroventricular or intrathecal injection.

18. (canceled)

19. (canceled)

20. A kit comprising the nucleic acid construct of claim 1, a viral vector comprising said nucleic acid construct, or a pharmaceutical composition comprising said viral vector of, and instructions for use thereof.
